(19) United States
(12) Patent Application Publication
(10) Pub. No.: US 2009/0093746 A1
(43) Pub. Date: Apr. 9, 2009

(54) APPLICATION OR CLEANING STICK, CONSISTING OF AT LEAST ONE TUBULAR BODY DESIGNED TO CONTAIN AT LEAST ONE LIQUID

(76) Inventor: Hakim Nacer, Grabels (FR)

Correspondence Address: EGBERT LAW OFFICES, 412 MAIN STREET, 7TH FLOOR, HOUSTON, TX, 77002, US

(21) Appl. No.: 12/281,852

(22) PCT Filed: Jan. 22, 2007
(86) PCT No.: PCT/FR2007/050670

§ 371 Date: Oct. 20, 2008

(30) Foreign Application Priority Data
Mar. 7, 2006 (FR) 0650791

(51) Int. Cl. A61M 35/00

(52) U.S. Cl. 604/3; 604/2

(57) ABSTRACT

The invention concerns an application or cleaning stick including at least one tubular body, capable of containing at least one liquid to flow through one first open end. The tubular body is closed at the second end, having a cleavable zone proximate the latter on which it includes an applicator tip. The tip extends well above the cleavable zone on the side of the second end and towards the first, protecting the cleavable zone after it has been ruptured and maintaining securely the second end to the tubular body after rupture. The tip is pre-colored with at least a color different from that of the tip of the first end of the body.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

This invention relates to an application or cleaning stick, consisting of at least one tubular body designed capable of containing at least one liquid aimed at flowing through a first open end, said tubular body being closed at its other end, referred to as second end, and including a cleavable zone proximate this second end, and including an applicator tip at least on said second end.

This invention falls within the field of the hygiene products for external use.

The invention relates in particular to the sticks used under the general name of cotton swabs, provided, at one or both of their ends, with applicator tips made out of absorbent or dressing material, such as absorbent cotton, gauze or the like.

Depending on the texture of the absorbent material, such a cotton stick or swab can be used for cleaning wounds, cleaning an area before its treatment, application of liquid or viscous products, make-up removing, cleaning of hollow conduits, or the like. Such applications are known in cosmetic, paramedic, veterinary use, even for precision works, such as for example precision mechanics or watch-making.

2. Description of Related Art Including Information Disclosed Under 37 CFR 1.97 and 37 CFR 1.98

For depositing or applying a liquid onto a determined area, there are known hollow sticks containing a liquid, for example a disinfectant or a cleaning product. This liquid is aimed at being deposited, or/and impregnating an applicator made out of absorbent material located at a first end of the stick.

At the second end of the stick opposite this first end, this liquid is stopped by a plug designed capable of ensuring maintaining the product inside the stick, or by a particular embodiment of this end, through sealing, melting, welding, gluing, pinching or the like.

In the vicinity of this second end, the stick includes a cleavable zone, permitting, in the way of a glass bulb a first end of which is already open, to cause the liquid it contains to flow through this first end when the second end is opened and put under atmospheric pressure.

An improvement consists in interposing, between the liquid contained in the stick and the first open end, a liquid plug permitting a progressive transfer of the liquid to the first end.

The tips, which such cotton sticks are usually provided with, about the first and the second end, are thus not aimed at being used in the same way. The one arranged at the second end is superimposed to the cleavable zone and is aimed at being handled by the user in order to perform the cleaving permitting the liquid contained in the stick to flow to the first end. The latter is then generally provided with a tip aimed at permitting the application of this liquid.

The problem for the user resides in marking the end to be cleaved and, on the other hand, the one that is aimed at being humidified by the liquid contained in the hollow stick.

It has been devised to mark to spot to be cleaved by means of an indicator, such as a colored ring, namely in the form of a ring with a printed color, namely in US2005/0171462. This printing cannot occur prior to coating the stick with the absorbent end material. The printing by ink jet or the like on the stick provided with the absorbent material has the drawback of staining the thus formed applicator, either through ink projection or through spreading of the dye into the absorbent material. Achieving a printed ring perfectly clean, without stains, is expensive and difficult. In addition, the visibility of such a printed ring is low.

Alternatively, placing an inserted part, such as a ring, proves expensive in comparison with the extremely low production cost price of such cotton sticks, manufactured in very large quantities, due to the addition of both an additional operation during the production process and an additional component to the product.

Moreover, in the event of placing a simple ring proximate the cleavable zone, such a ring cannot be placed directly in front of the cleavable zone, so that the user can be inclined to rupture the stick, not at the level of the cleavable zone, but at the level of the indicator ring.

There is a risk for the operator to rupture the stick improperly, even to get injured, because of the creation of sharp portions when rupturing the stick. This is particularly prohibitory for all uses related to hygiene, namely in a sterile or highly clean environment.

According to FR 2 851 180, it has also been devised to provide a narrowed area, for example in the form of a circular groove, in the thickness of the absorbent material, and in front of the cleavable zone of the stick. In this case too, this operation represents an extra cost, and the marking of this narrowed area proves little visible.

Likewise, providing a protruding area, as known from US2005/0171462, has the same costs and drawbacks.

In short, not only is this area not immediately visible for the user, but, in addition, for a person, who has no perfect visual acuity, it is perfectly invisible.

In the known embodiments, one also observes that, after rupturing the cleavable zone, the tip separates from the stick, so that the rupture is visible, and could cause an injury to the user. If the rupture is foreseen only partially, this does not solve the problem, since the applicator tip does not hide this rupture. Even worse, maintaining the ruptured tip integral with the stick can cause the user to be inclined to use it, hence there is also a risk for the latter to get injured by the visible cleaved portion.

One also observes that such an applicator is a mass-produced product, in particular, in an cosmetic, paramedical or hospital environment, and that, despite its relatively low purchase price per unit, using only one tip out of the two a stick includes represents a waste that should be avoidable.

Finally, it should be noted that the user has not always his two hands available for cleaving the stick, either due to cosmetic, hygiene or paramedic care to be applied as a matter of fact to one of both hands or due to the non-availability of one of both hands for holding an organ, or handling another instrument or another product.

In brief, the main problem for the operator resides in instantaneously marking the end to be cleaved. The design of the stick should be such that he can perform this cleavage namely with one hand, without having to look, at the level of this end, for the accurate location of the cleavable zone in order to exert an accurate mechanical action in this extremely small area. Now, the prior art documents designate by a thread, or a groove, or a rim, always with a very small width, the exact location of the cleavable zone.

### BRIEF SUMMARY OF THE INVENTION

The invention is aimed at coping with the drawbacks of the state of the art by providing a reliable and economical solution for these problems, by putting at the disposal of the operator an application or cleaning stick easy to be cleaved, with an easy marking of the end to be cleaved, without any risk of a bad cleavage, or injury, easy to be handled with one hand, and permitting to use both applicator tips, provided with absorbent material, it may include.

This application or cleaning stick according to the invention has, in addition, an immaculate aspect, due to the absence of any staining through printing or the like, and permits, in a simple and cheap way, to identify, as the case may be, the nature of the product contained in the body of the stick, or/and the nature of the absorbent material forming either tip, and thus makes its use particularly user-friendly for the operator, especially in an environment in which he has to handle daily all kinds of products.

To this end, the invention relates to an application or cleaning stick, consisting of at least one tubular body designed capable of containing at least one liquid aimed at flowing through a first open end, said tubular body being closed at its other end, referred to as second end, and including a cleavable zone proximate this second end, and including an applicator tip at least on said second end. The applicator tip on the second end is pervious to air and extends well above the cleavable zone on the side of this second end and towards the first end, so as to constitute means for protecting said cleavable zone after it has been ruptured, and to constitute means for maintaining the second end integral with said tubular body after the rupture of said cleavable zone, and said applicator tip on the second end includes identification means.

According to a feature of the invention, this applicator tip on the second end is pre-colored in at least one color different from that or those of the eventual applicator tip arranged on the first end of said tubular body.

According to a feature of the invention, said tubular body includes, on both sides of said cleavable zone, coupling means designed capable of cooperating with the material constituting said applicator tip on the second end and of constituting said means for maintaining said second end integral with said tubular body after rupturing said cleavable zone.

According to a particular feature, said coupling means are formed by an emboss provided for on said tubular body.

According to another feature of the invention, said applicator tip on the second end extends, on each side of said cleavable zone, over a length at least equal to the diameter of said tubular body, in order to constitute said means for protecting said cleavable zone after rupturing same.

According to a feature of the invention, such an applicator tip is formed of an absorbent material such as absorbent cotton, gauze or the like.

According to another feature of the invention, the rupture at the level of the cleavable zone is a partial rupture.

According to yet another feature of the invention, said applicator tip located on said second end is designed capable of being seized between at least two fingers, on the side between the cleavable zone and the second end. The cleavable zone is designed capable of rupturing under the action of at least another finger resting on the tubular body, on the side of the first end, or vice-versa.

According to another feature of the invention, the first end of the application stick is also provided with an applicator tip.

Further features and advantages of the invention will become clear from the following detailed description of the non-restrictive embodiments of the invention, with reference to the attached drawings.

### BRIEF DESCRIPTION OF THE SEVERAL VIEWS OF THE DRAWINGS

FIG. 1 is a schematic view of an application or cleaning stick according to the invention.

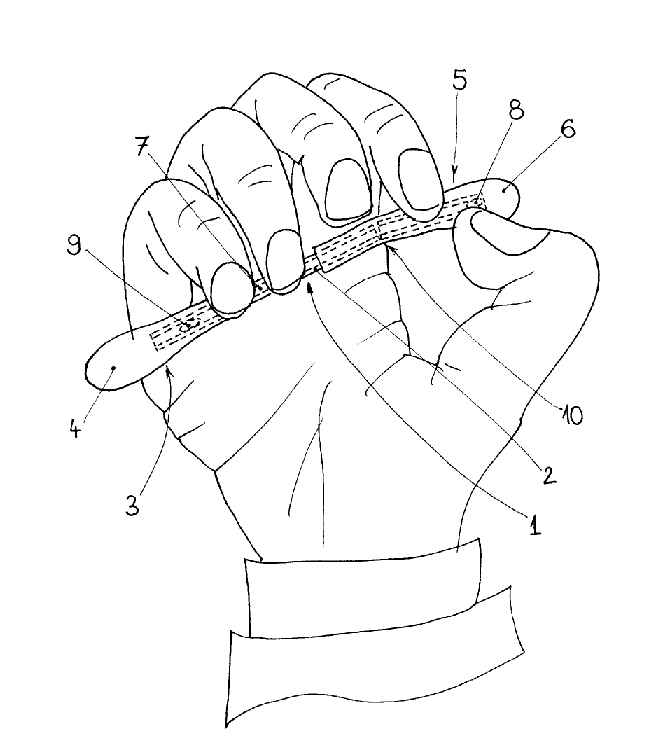

### DETAILED DESCRIPTION OF THE INVENTION

The invention falls within the field of the hygiene products for external use.

The invention relates in particular to an application or cleaning stick 1.

As can be seen in the single figure, the application or cleaning stick 1 includes a tubular body 2. The latter includes a first open 3. This first end 3 can be provided with an applicator tip 4, made out of absorbent or dressing material, such as absorbent cotton, polyester, cotton-polyester mixture, gauze or the like. It can also be provided with an accessory such as a flat brush, a brush, a sponge, a foam form, or a large-diameter cotton ball, or the like.

It is obvious that an applicator tip of an application or cleaning stick according to the invention can of course be used both for applying product and for removing product, thanks to the absorbent properties of the material forming this tip.

The tubular body 2 is provided, at a second end 5, with an applicator tip 6.

Depending on the use for which the application stick 1 is aimed, this applicator tip 6, usually of an ellipsoidal shape, can also be of a conical, spherical shape or the like.

This also applies for the eventual applicator tip 4 on the first end 3.

The tubular body 2 is designed capable of containing a liquid 7.

To this end, the tubular body 2 is closed, on the side of its second end 5, by a permanent closing means 8, such as a plug, or also by welding or gluing of the tubular body 2 on itself or to a foreign body, or the like.

The liquid 7 is maintained in the tubular body 2, only part of which it occupies, and has a surface tension such that it does not flow out and remains confined within the tubular body 2 under the action of the atmospheric pressure.

The liquid 7 is advantageously confined, on the side of the first end, by means of a phase 9 of liquid or viscous material, with a surface tension and a viscosity different from those of the service liquid, forming a barrier, in order to avoid pollution or evaporation of same, and referred to as liquid plug.

The tubular body 2 includes a less resistant cleavable zone 10 designed capable of being ruptured when applying a manual force exerted with a component perpendicular to the axis of the tubular body 2, proximate the cleavable zone 10.

The applicator tip 6 on the second end is pervious to air and extends well above the cleavable zone 10, on the side of this second end 5 and towards the first end 3.

The applicator tip 6 extends above the cleavable zone 10, in a way capable of:
constituting means for protecting this cleavable zone 10 after rupturing same, so as to avoid any injury or pollution;
constituting means for maintaining the second end 5 integral with the tubular body 2 after rupturing the cleavable zone 10; and
constituting means for identifying the end of the stick 1 including the cleavable zone 10; to this end, the applicator tip 6 on the second end 5 is pre-colored in a color different from that of the applicator tip 4 arranged on the first end 3 of the tubular body 2.

In a preferred application, the tubular body 2 includes, on both sides of the cleavable zone 10, coupling means 11.

These coupling means 11 are designed capable of cooperating with the material forming the applicator tip 6 on the second end. The coupling means 11 constitute said means for maintaining the second end 5 integral with the tubular body 2 after rupturing the cleavable zone 10.

In a particularly advantageous and economic way, these coupling means 11 are formed by an emboss provided for on the tubular body 2, for example under the action of sharp or heating tools, or deforming, knurling tools, or the like. This emboss can namely adopt the shape of rotation or non-rotation kerns, or also of rotation, slanted grooves or/and rims or the like. Preferably, this emboss is made by causing the tubular body 2 to rotate, and bringing the latter into contact with one or several tools arranged perpendicular or/and oblique with respect to its axis of rotation.

In a preferred embodiment of the invention, the applicator tip 6 on the second end 5 extends, on each side of the cleavable zone 10, over a length at least equal to the diameter of the tubular body 2, in order to constitute the means for protecting the cleavable zone 10 after rupturing same.

In a preferred embodiment, the material forming the applicator tip 6 on the second end 5 is colored in the mass, downstream of its placing on the tubular body 2. In the case of using cotton, which is usually packaged in bales of several hundred kg, the cotton will thus be colored at the scale of the bale, or even more bales. In a preferred embodiment, this applicator tip 6 is pre-colored in a color different from the one that characterizes it in the natural state.

This solution permits to provide homogenous and perfectly regular coloring, thanks to the control of all the physical-chemical parameters during the treatment of the bale.

All the a posteriori coloration tests of an applicator tip 6 already applied on an application stick 1 indeed show a color change, since each stick 1 is stored under own conditions, namely temperature and hygrometry conditions.

The pre-coloring in the mass of the bale has also the advantage of saving coloring material, as well as several expensive process operations: placing on a trolley, printing or coloring, drying.

In particular, an applicator tip 6 made out of absorbent cotton will be colored in a color different from white.

In order to use the applicator or cleaning stick 1, the operator takes it in his hand, identifies, by its color, the second end 5, which he maintains at a height higher than that of the first end 3, and brings it close to the area to be treated.

The operator seizes the second end 5 and causes it to perform a relative motion with respect to the tubular body 2, by applying to same a force with a component perpendicular to the axis of the latter. He can thus trigger the rupture of the cleavable zone 10.

When rupturing the cleavable zone 10, putting the liquid 7 under atmospheric pressure, on the side of the second end 5, allows the liquid 7 to flow by gravity towards the first end 3 and the applicator tip 4 it is provided with. The liquid 7 then humidifies the applicator tip 4 and makes it suitable for being used.

In this respect, the selection, for the applicator tip 4, of a polyester-cotton mixture permits a slow absorption of the liquid product 7 contained in the tubular body 2, which is advantageous.

The texture of the applicator tip 6 of the second end 5 and a large covering of the latter on the cleavable zone 10 permit to maintain it integral with the tubular body 2.

In a preferred embodiment, a very tight rolling-up, coiling or spinning of the applicator tip 6 about the tubular body 2 on both sides of the cleavable zone 10 ensures such a maintaining, with a small angular shift with respect to the axis of the tubular body 2. This very tight spinning also provides an important saving of coating material forming the applicator tip. The operator can thus use the applicator tip 6 in addition to the applicator tip 4 through which the liquid 7 flowed out.

The applicator tip 6 of the second end 5 can, like the applicator tip 4 of the first end 3, adopt particular shapes. The coiling or spinning process namely permits to easily make zones of different cross-section, in order to improve the gripping, or any other use.

This embodiment ensures that the surfaces resulting from the rupture of the cleavable zone 10 are maintained under the applicator tip 6, and the operator is thus protected against any injury caused by an eventual chip generated during the rupture. Likewise, this arrangement permits to avoid any injury to the area on which the applicator tip 6 has to move 6.

In a preferred embodiment, the rupture of the cleavable zone 10 is a partial rupture, so as to contribute to maintaining the second end 5 integral with the tubular body 2 after rupturing the cleavable zone 10.

In this respect, the tight coiling of the absorbent material, such as cotton, about the tubular body 2, contributes to the stiffness of the application stick 1 according to the invention, and permits reducing the thickness of this tubular body 2, proportionally to its diameter, which results, on the one hand, into an important saving of material and, on the other hand, into an increased capacity of liquid for a same outer diameter. The latter advantage is particularly interesting for the application sticks 1 for veterinary use, which are of a larger size than the usual sticks, and for which one wants to have a larger available capacity.

The cleavable zone 10 is preferably made in the form of a reduced cross-section, such as one, or several, partially or fully rotation groove. For the same diameter of the tubular body, and the same depth of such a groove, the remaining cross-section, when the thickness of the tubular body 2 is reduced, is reduced accordingly, which results into an increased easiness for the operator to perform the rupturing of the cleavable zone 10.

In order to facilitate the handling by the operator, namely with one hand, as shown in the single figure, the applicator tip 6 located at the second end 5 is designed capable of being seized between at least two fingers, on the side between the cleavable zone 10 and the second end 5. The cleavable zone 10 is designed capable of rupturing under the action of at least another finger resting on the tubular body 2, on the side of the first end 3, or vice-versa.

Many drugs or products for various cosmetic, veterinary, esthetical (hardening glue for mails, solvent, dyestuff for hair), technical (quickly drying glue) applications or the like require a blending of several substances immediately before application, in order to be able to preserve the integral properties of the molecules or active agents being part of the formula.

In a particular embodiment, not shown in the figure, the application stick 1 includes a plurality of primary tubular bodies 2A. These primary tubular bodies 2A can, as the case may be, be inserted into each other, or be juxtaposed in a substantially parallel way, in order to form a bundle, or also be assembled by combining these two ways, i.e. in the form of a bundle comprising two tubes inserted into each other, or of one tube into which is inserted at least one such bundle.

Each of these primary tubular bodies 2A is capable of containing a particular primary liquid 7A, and includes a cleavable zone 10A. The juxtaposition of these primary tubular bodies 2A is performed so as to juxtapose their cleavable zones 10A, in order to form one single rupturing zone at the level of the stick 1, so that the operator has to perform only one rupturing operation, like when he handles one single tubular body 2.

Preferably, these primary tubular bodies contain different liquids or products, which are blended, during the rupture, at the level of the first end 3 of the stick 1, or of the tip 4 this very first end 3 eventually includes. Each primary tubular body contains the right quantity necessary to obtain a blending according to the exact proportions, which are very often highly accurate, in particular in the case of adhesives.

Such an application according to the invention permits, by packaging small quantities, to avoid a frequent waste when preparing multi-component mixtures made from different packaging, often in excess, part of which cannot be used, and of course copes with the measuring problem, which is always difficult to be performed exactly and with cleanness in this type of mixing.

In yet another embodiment, not shown in the figure, one and the same tubular body 2 contains several different liquid products 7, aimed at being mixed after rupturing the cleavable zone 10. These liquids are then separated from each other by means of phases of liquid or viscous materials similar to the one 9 described above for preserving the liquid 7, referred to as liquid plugs, and preferably formed of silicone material.

In a preferred embodiment, the tubular body 2 is made out of a semi-translucent material, so as to protect the photosensitivity of the liquid 7 against ultraviolet rays and the like, and to ensure its good preservation in time.

In a variant embodiment, the tubular body 2 of the stick is colored, or also provided with a labeling or a marking identifying the liquid it contains, in order to facilitate the identification of the latter.

The user can thus have different color combinations between the tubular body 2 and the applicator tip 6. Each combination corresponds to a particular application. The different combinations are shown in a table. The identification of the stick 1 is thus easy and immediate. The control is facilitated.

It is obvious that, each time a liquid is evoked in the preceding description, this also refers to a product in powder capable of flowing in a tubular body.

Of course, the invention is not limited to the examples shown and described above, which can have variants and modifications without departing from the framework of the invention.

1. Application or cleaning stick comprising:
at least one tubular body containing at least one liquid flowing through a first open end, the tubular body being closed at a second end and having a cleavable zone proximate to said second end and an applicator tip on said second end, wherein said applicator tip is pervious to air and extends above said cleavable zone on a side of said second end and towards said first end said cleavable zone being protected after being ruptured, said second end being maintained integral with the tubular body after rupture of said cleavable zone, said applicator tip being pre-colored in a color different from a tip of the first end of the tubular body.

2. Application or cleaning stick, according to claim 1, wherein said tubular body includes, comprises:
a coupling means on both sides of said cleavable zone, being cooperative with material of said applicator tip on said second end, said second end being maintained integral with the tubular body after rupturing said cleavable zone.

3. Application or cleaning stick, according to claim 2, wherein said coupling means are formed by an emboss on the tubular body.

4. Application or cleaning stick, according to claim 3, wherein said emboss adopts a shape of kerns, or rotation or slanted grooves.

5. Application or cleaning stick according to claim 3, wherein said emboss is formed by causing the tubular body to rotate, and bringing the tubular body into contact with a tool arranged perpendicular or/and oblique with respect to an axis of rotation thereof.

6. Application or cleaning stick according to claim 2, wherein said applicator tip on the second end extends, on each side of said cleavable zone, over a length at least equal to the diameter of the tubular body, said cleavable zone being protected after rupturing.

7. Application or cleaning stick, according to claim 1, wherein rupture at said cleavable zone is a partial rupture.

8. Application or cleaning stick, according to claim 1, wherein said applicator tip is seized on sides between said cleavable zone and said second end, said cleavable zone rupturing under action of resting on the tubular body, on the side of the first end, or vice-versa.

9. Application or cleaning stick, according to claim 1, wherein said applicator tip is comprised of absorbent material.

10. Application or cleaning stick, according to claim 1, wherein the tubular body is colored or provided with a marking identifying the liquid contained therein.

11. Application or cleaning stick according to claim 1, wherein the photosensitivity of the liquid is protected by the tubular body against ultraviolet rays.

12. Application or cleaning stick according to claim 1, wherein said applicator tip is pre-colored in a color different from color in a natural state.

13. Application or cleaning stick according to claim 12, wherein pre-coloring is carried out in a mass of a bale.

14. Application or cleaning stick according to claim 1, wherein each combination of colors between said tubular body and said applicator tip is shown in a reference table for a particular application.

15. Application or cleaning stick according to claim 1, further comprising:
a plurality of primary tubular bodies, inserted into each other, and/or juxtaposed in a substantially parallel way, in order to form a bundle.

16. Application or cleaning stick according to claim 15, wherein said primary tubular bodies are each comprised of a cleavable zone, which are juxtaposed, in order to form one single rupturing zone.

17. Application or cleaning stick according to claim 16, wherein said primary tubular bodies contain different liquids.

18. Application or cleaning stick according to claim 16, wherein said same tubular body contains several different liquid products, said liquid being mixed after rupturing said cleavable zone and separated from each other by means of liquid plugs.
